(19) United States
(12) Patent Application Publication
(10) Pub. No.: US 2024/0238843 A1
(43) Pub. Date: Jul. 18, 2024

(54) METHODS AND SYSTEMS FOR A MULTI-FREQUENCY PIEZO-COMPOSITE TRANSDUCER ARRAY

(71) Applicant: GE Precision Healthcare LLC, Wauwatosa, WI (US)

(72) Inventors: Alexis Hubert, Valbonne (FR); Giandonato Stallone, Valbonne (FR); Edouard Da Cruz, Valbonne (FR); Manon Chateauvieux, Valbonne (FR); Solenne Drewniak, Valbonne (FR)

(21) Appl. No.: 18/155,004

(22) Filed: Jan. 16, 2023

(51) Int. Cl. B06B 1/06; H10N 30/086; H10N 30/088

(52) U.S. Cl. CPC B06B 1/0622; H10N 30/086; H10N 30/088; A61B 8/4488; B06B 2201/76

(57) ABSTRACT

Various methods and systems are provided for a multi-frequency transducer array. In one example, the transducer array includes a plurality of elements formed of two or more piezoelectric materials, the two or more piezoelectric materials having different resonance frequencies. Furthermore, the two or more piezoelectric materials may be oriented independent of a dicing pattern of the transducer array, the dicing pattern defining the plurality of elements.

### FIELD

Embodiments of the subject matter disclosed herein relate to a transducer for a medical device.

### BACKGROUND

Transducer probes are used in a variety of applications to convert energy from a physical form to an electrical form. For example, a transducer probe may include piezoelectric materials which generate electrical voltage from a mechanical stress or strain exerted on the materials. Piezoelectric transducer probes are configured to be highly sensitive to provide large signal amplitudes, broad bandwidth for use across a wide range of frequencies, and short-duration impulse for high axial resolution. Such properties are desirable for medical applications such as imaging, non-destructive evaluation, fluid flow sensing, etc. Furthermore, incorporation of more than one type of piezoelectric material into the transducer probe may allow the transducer probe to operate across a broad range of frequencies, thereby expanding its applicability.

### BRIEF DESCRIPTION

In one embodiment, a transducer array comprises a plurality of elements formed of two or more piezoelectric materials, the two or more piezoelectric materials having different resonance frequencies. The two or more piezoelectric materials may be oriented independent of a dicing pattern of the transducer array, the dicing pattern defining the plurality of elements. In this way, a multi-frequency transducer probe may be provided and manufactured via a low cost, efficient process.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example of an acoustic stack of an ultrasound transducer.

FIG. 2 shows an example of a homogeneous multi-element transducer array.

FIG. 3 shows an example of a 1D array of a transducer formed of a piezo-composite.

FIG. 4 shows an example of a 1.25D array of a transducer formed of a piezo-composite.

FIG. 5 shows an example of a matrix 2D array for a transducer formed of a piezo-composite.

FIG. 6 shows an example of side view of a transducer array formed of a piezo-composite.

FIG. 7 shows the piezo-composite of FIGS. 3-6 with a pitch indicated.

FIG. 8 shows an example of a piezo-composite with a different proportion of elements relative to the piezo-composite of FIG. 7.

FIG. 9 shows an example of a piezo-composite with a different kerf width relative to the piezo-composite of FIG. 7.

FIG. 10 shows an example of a piezo-composite with a different array angle relative to the piezo-composite of FIG. 7.

FIG. 11 shows an example of a piezo-composite with variable element width.

FIG. 12 shows a first example of a piezo-composite stack before grinding.

FIG. 13 shows a second example of a piezo-composite stack before grinding.

FIG. 14 shows a final structure of the piezo-composite stack of FIG. 13.

FIG. 15 shows an example of dicing and singulation of a piezo-composite wafer formed of the piezo-composite stack of FIG. 14.

FIG. 16 shows an example of a method for fabricating a piezo-composite transducer array.

FIG. 17 shows an example of a piezo-composite stack that precludes grinding.

### DETAILED DESCRIPTION

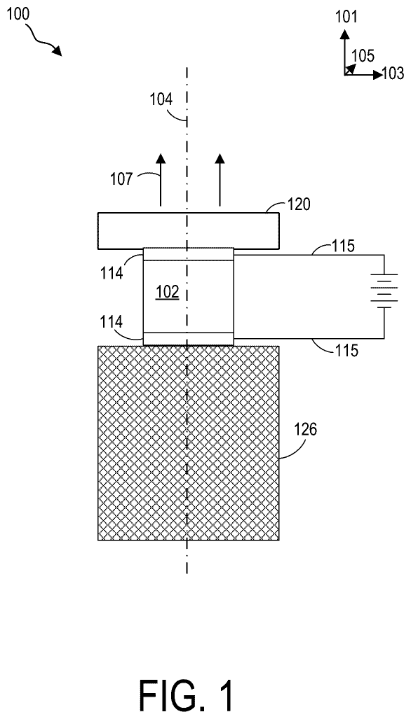

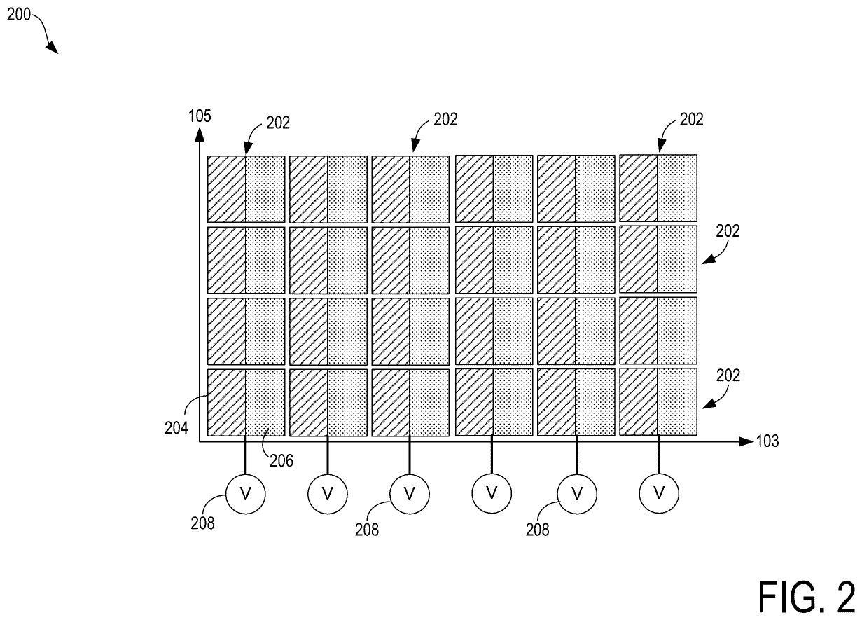

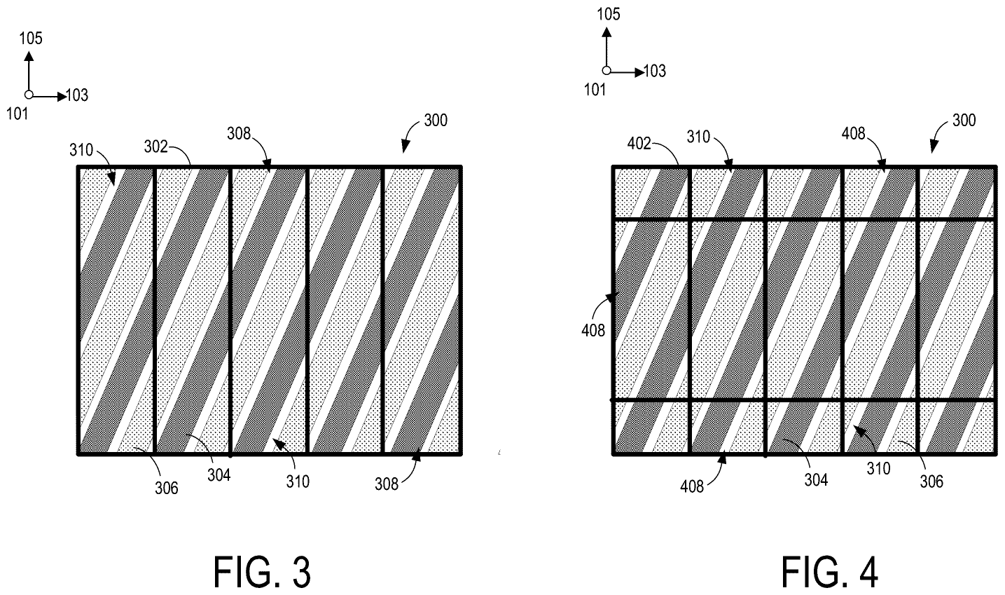

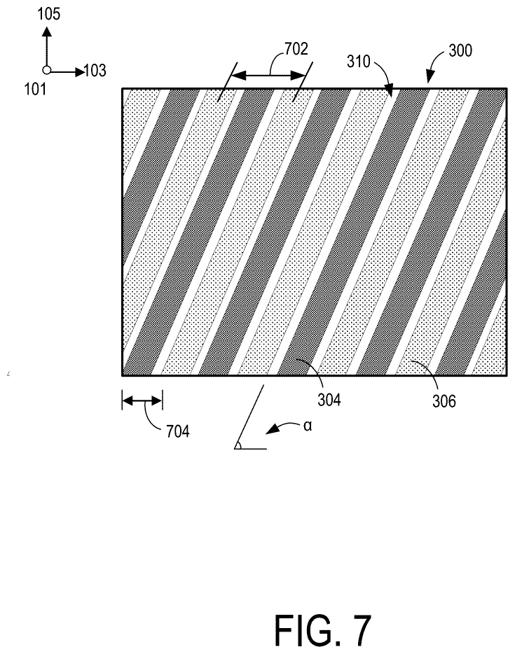

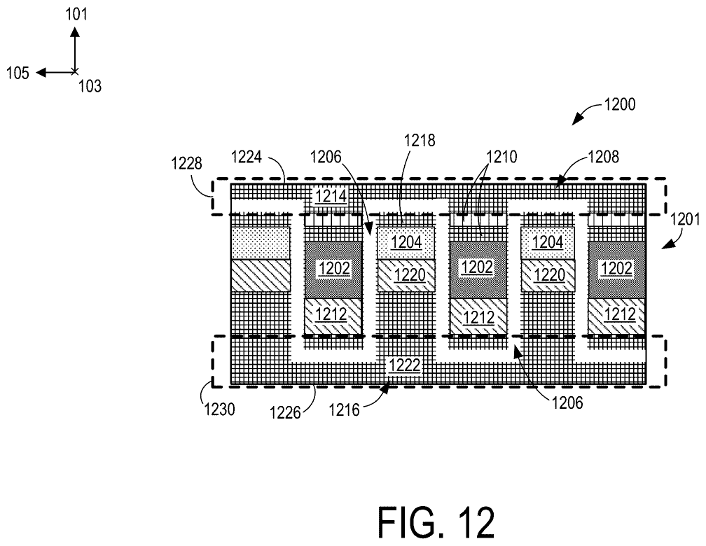

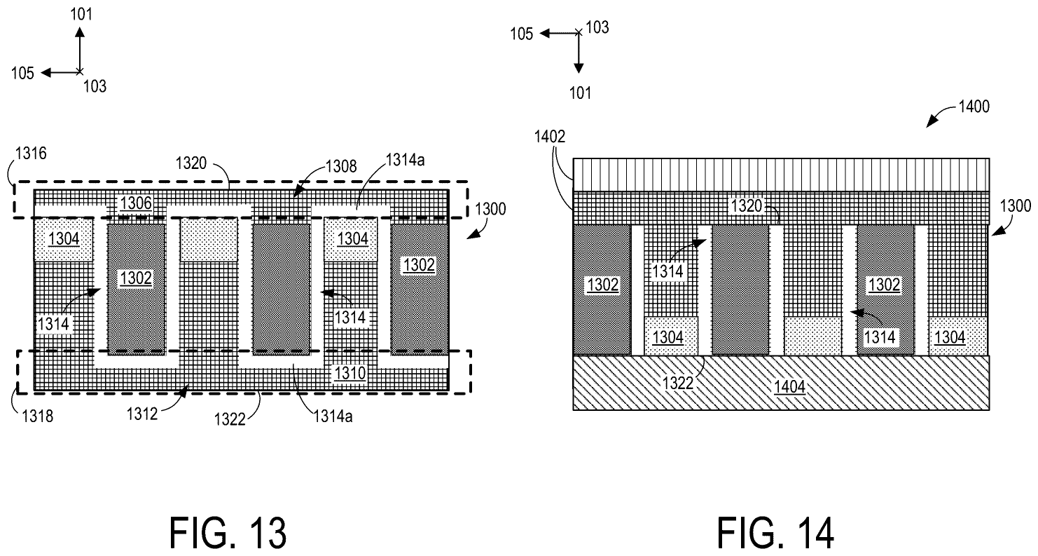

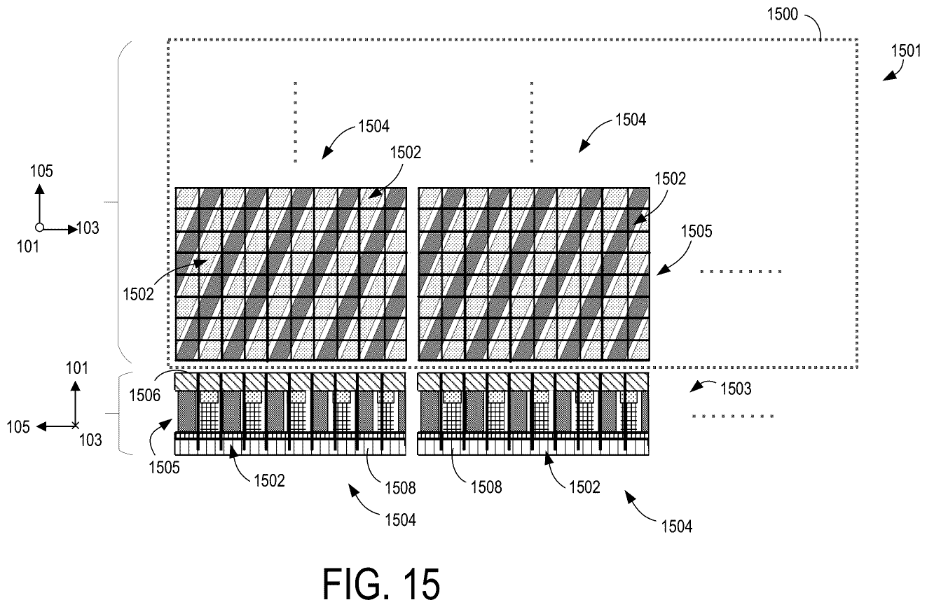

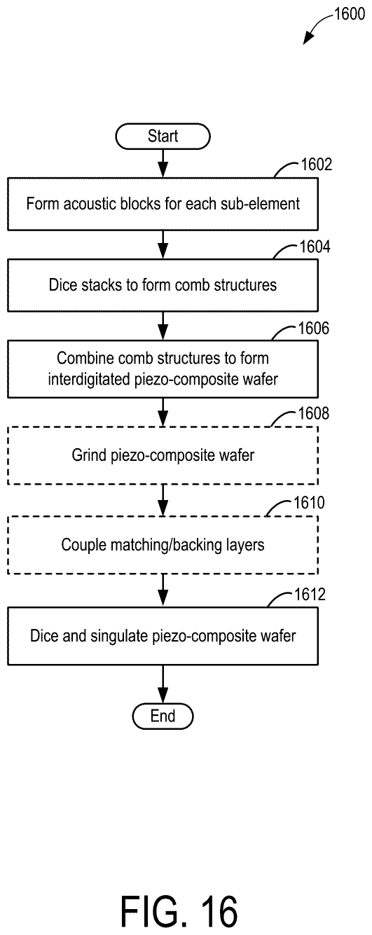

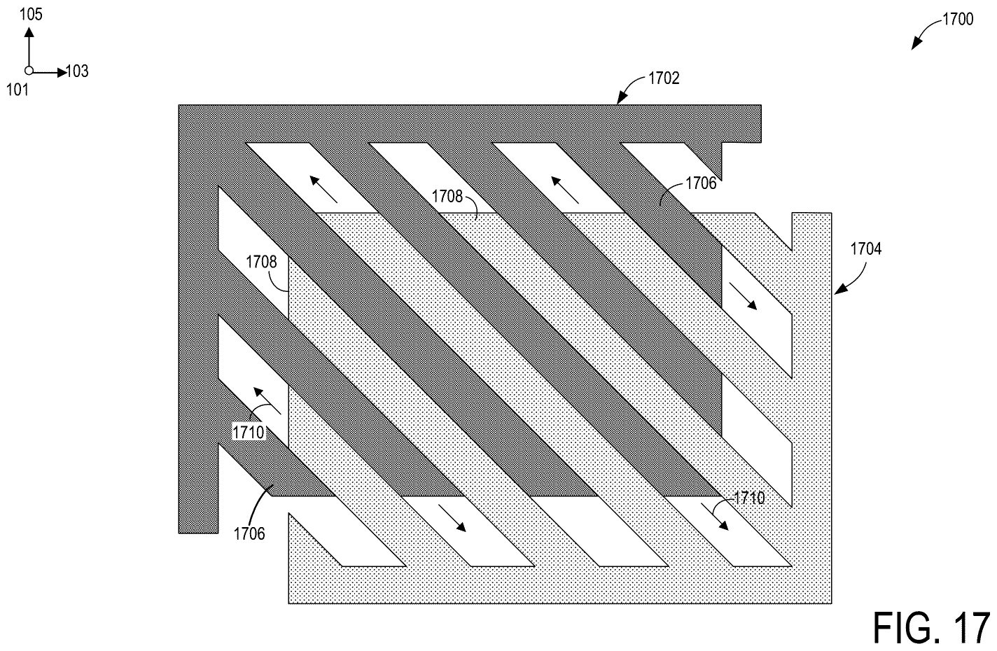

The following description relates to various embodiments of an acoustic stack for a transducer probe. The acoustic stack may be configured with a broad frequency bandwidth that may be tuned by adapting the acoustic stack with a piezoelectric element formed from more than one sub-element. An example of an acoustic stack for a transducer probe is shown in FIG. 1. Each of the more than one sub-element may be a different type of element with a different resonance frequency. Relative proportions of the more than one sub-element may be maintained constant along both an azimuth direction and an elevation direction of the transducer probe to form a homogeneous array. An example of a homogeneous multi-frequency transducer array is depicted in FIG. 2, which may demand alignment of an array pattern (e.g., a pattern upon which dicing of the array into elements is based) with an orientation of the transducer array sub-elements. In the embodiments described herein, a transducer array formed of a piezo-composite, the piezo-composite formed of at least two different piezoelectric materials or sub-elements, may be configured such that the orientation of the array or dicing pattern does not dictate the alignment of the sub-elements. Examples of transducer arrays that may be formed from the piezo-composite based on a dicing pattern are shown in FIGS. 3-5 from a top-down view, and in FIG. 6 from a side view. An acoustic performance of the piezo-composite may be controlled by tuning various parameters (e.g., physical characteristics) of the piezo-composite, such as a pitch of sub-element of the piezo-composite, a relative proportion of sub-elements, a kerf width, a sub-element angle, and variations in kerf width and/or sub-element pitch within a given piezo-composite, as shown in FIGS. 7-11, respectively. Examples of acoustic stacks formed of piezo-composites, shown prior to grinding, are illustrated in FIGS. 12-13, and an example of a structure of a final piezo-composite stack, after fabrication is complete, is depicted in FIG. 14. As shown in FIG. 15, the final piezo-composite stack may be singulated from a wafer into individual transducer arrays. An example of a method for manufacturing a transducer array from a piezo-composite is shown in FIG. 16. An example of an acoustic stack formed of a piezo-composite that precludes grinding is shown in FIG. 17.

FIGS. 1-15, and 17 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

Piezoelectric elements may be implemented in transducer probes for a wide range of medical applications, including imaging, non-destructive testing, diagnosis, measuring blood flow, etc. The piezoelectric elements may be formed of a class of crystalline materials that become electrically polarized when subjected to a mechanical strain. When stressed, the piezoelectric elements output a voltage that is proportional to the applied stress.

A piezoelectric transducer probe, e.g., a device utilizing a piezoelectric effect to convert energy from one form to another, may offer high sensitivity, high frequency response and high transient response. In some examples, such as in ultrasound transducer probes, a converse piezoelectric effect may be leveraged where electricity is applied to the piezoelectric elements, causing deformation of the material and generation of ultrasonic waves. As such, an external, mechanical force is not demanded and the piezoelectric transducer probe may be packaged as a compact, easily transportable device.

Although the piezoelectric transducer probe is a highly sensitive instrument, an operational frequency bandwidth of the probe may be narrow. For example, the piezoelectric material may be associated with a low frequency, e.g., between 0.5-2.25 MHz, or a high frequency, e.g., between 15.0-25.0 MHz, but not both. Similarly, the transducer probe may be adapted for transmitting or receiving but may not be equipped for high performance in both applications due to a focused frequency range of the particular type of piezoelectric material. Broadband transducer probes may provide wider operational frequency ranges but adapting the probes with electrical impedance matching may be challenging and cost prohibitive.

In one example, the issues described above may be addressed by a piezoelectric transducer probe adapted with a multi-frequency transducer array. The multi-frequency transducer array may include a plurality of elements formed of two or more piezoelectric materials or sub-elements, e.g., a piezo-composite, with different respective operating frequencies. The two or more piezoelectric material types may be arranged at an angle relative to an azimuth direction of the transducer array. Furthermore, kerf width, sub-element angle, and sub-element pitch, may be varied and optimized to adjust an acoustic performance of the transducer array and decrease a homogeneity of element compositions. By configuring the plurality of elements with different relative proportions of the two or more piezoelectric materials, a range and continuity of frequencies of the plurality elements may be increased. An acoustic stack comprising the piezo-composite may be incorporated into transducer probes across a variety of applications and provide high resolution with reduced ringdown, as well as controlled frequency variation across the transducer array to facilitate an averaging effect. Additionally, fabrication of the transducer array may be achieved with low front end labor, scalability, and greater flexibility with respect to volume-to-price constraints, via a wafer approach.

By fabricating the piezo-composite transducer array from the wafer approach, various transducer arrays including 1D, 1.25D, 1.5D, 1.75D, and/or matrix element arrays may be formed from 2-2 piezo-composite structures. The transducer arrays may have multi-frequency properties without conforming to a demand for alignment between a pattern of piezo-composite and a dicing pattern of the array along both elevation and azimuth directions of the transducer array. The piezo-composite may provide additional control over a structure of the transducer acoustic stack by enabling variation in a width and a pitch of the sub-elements that is independent of dimensions of the plurality of elements defined by the dicing patterns, which may increase suppression of lateral modes. The composition of the plurality of elements (e.g., proportions of sub-elements) may be tuned according to a desired acoustic performance, allowing broadband operation to be achieved while maintaining production costs low.

The wafer approach also allows multiple transducer probes to be constructed from a common wafer. The piezo-composite may be formed from different types of materials, in addition to piezoelectric transducer elements of different frequencies, such as single crystal materials. A resulting transducer probe may be utilized as a one probe solution for both therapy (e.g., at low frequency) and imaging (e.g., at high frequency). The transducer probe may further be optimized for harmonic imaging where operation at low frequency may be optimized for signal transmission and operation at high frequency may be optimized for signal reception. Further, multi-frequency capabilities of the piezo-composite transducer probe may provide a one probe solution for Point of Care via broadband operation that addresses multiple applications concurrently. Further details of piezo-composite arrays which may provide the aforementioned benefits are elaborated further below.

Multi-frequency piezoelectric transducers, as described herein, may be used in a variety of medical devices. For example, as shown in FIG. 1, a piezoelectric transducer may be included in an ultrasound probe used to create an image based on ultrasonic signals. It will be appreciated that the ultrasound probe is a non-limiting example of a medical device utilizing the piezoelectric transducer and incorporation of the piezoelectric transducer in other medical devices has been envisioned. For example, the piezoelectric transducer may be used to convert energy in non-destructive testers, Jetter systems, high voltage power sources, etc. The following description of FIG. 1 is an exemplary overview of how the piezoelectric transducer may be implemented in the ultrasound transducer probe.

An ultrasound probe includes one or more active components for generating an ultrasonic signal. An example of an active component, or piezoelectric element 102 of an ultrasound probe is shown in a schematic diagram of an acoustic stack 100 in FIG. 1, with a central axis 104. A set of reference axes are provided, indicating a propagation direction 101 (e.g., wave propagation), an azimuth direction 103, and an elevation direction 105. In other examples, the set of reference axes may represent a z-axis 101, an x-axis 103, and a y-axis 105. The piezoelectric element 102 is shown in FIG. 1 with the central axis 104 aligned parallel with the propagation direction 101.

It will be noted that while the acoustic stack 100 is shown configured for a linear ultrasound probe and the propagation direction is described as parallel with the z-axis in FIG. 1, other examples may include a propagation direction that is angled relative to the z-axis, depending on a shape of a piezoelectric element array. For example, the ultrasound probe may be curvilinear or phased array, thus generating non-linear beams that are not parallel with the z-axis.

While a single piezoelectric element is shown in FIG. 1, the ultrasound probe may include a plurality of piezoelectric elements arranged in an array and individually coupled to an electrical energy source by wires. Each electrical circuit formed of one or more piezoelectric elements may be a transducer. In some examples, the transducer may include an array of piezoelectric elements which may arranged in a variety of patterns, or matrices, including one-dimensional (1D) linear, two-dimensional (2D) square, 2D annular, etc. In one example, the transducer may be formed from more than one type of piezoelectric element, thereby providing a multi-frequency piezoelectric transducer. A frequency distribution along each of the azimuth and elevation directions may adapted to be uniform or non-uniform. Further details of the multi-frequency piezoelectric transducer are provided below, with reference to FIGS. 3-16.

Each transducer may be electrically insulated from adjacent transducers but may all be coupled to common layers positioned above and below the piezoelectric element, with respect to the propagation direction. The plurality of piezoelectric elements and accompanying layers may be enclosed by an outer housing of the ultrasound probe, which may be, for example, a plastic case with a variety of geometries. For example, the outer housing may be a rectangular block, a cylinder, or a shape configured to fit into a user's hand comfortably. As such, components shown in FIG. 1 may be adapted to have geometries and dimensions suitable to fit within the outer housing of the ultrasound probe.

The piezoelectric element 102 may be a block formed of a natural material such as quartz, or a synthetic material, such as lead zirconate titanate, that deforms and vibrates when a voltage is applied by, for example, a transmitter. In some examples, the piezoelectric element 102 may be a single crystal with crystallographic axes, such as lithium niobate and PMN-PT (Pb(Mg_{1/3}Nb_{2/3})O₃—PbTiO₃). The vibration of the piezoelectric element 102 generates an ultrasonic signal formed of ultrasonic waves that are transmitted out of the ultrasound probe in a direction indicated by arrows 107, e.g., along the propagation direction 101. The piezoelectric element 102 may also receive ultrasonic waves, such as ultrasonic waves reflected from a target object, and convert the ultrasonic waves to a voltage. The voltage may be transmitted to a receiver of the ultrasound imaging system and processed into an image.

Electrodes 114 may be in direct contact with the piezoelectric element 102 to transmit the voltage via wires 115, the voltage converted from the ultrasonic waves. The wires 115 may be connected to a circuit board (not shown) to which a plurality of wires from electrodes of the plurality of piezoelectric elements may be fixed. The circuit board may be coupled to a coaxial cable providing electronic communication between the ultrasound probe and the receiver.

An acoustic matching layer 120 may be arranged above the piezoelectric element 102, with respect to the propagation direction 101, oriented perpendicular to the central axis 104. The acoustic matching layer 120 may be a material positioned between the piezoelectric element 102 and a target object to be imaged. By arranging the acoustic matching layer 120 in between, the ultrasonic waves may first pass through the acoustic matching layer 120, and emerge from the acoustic matching layer 120 in phase, thereby reducing a likelihood of reflection at the target object. The acoustic matching layer 120 may shorten a pulse length of the ultrasonic signal, thereby increasing an axial resolution of the signal.

A backing (e.g., backing layer) 126 may be arranged below the piezoelectric element 102, with respect to the propagation direction 101. In some examples, the backing 126 may be a block of material that extends along the azimuth direction 103 so that each of the plurality of piezoelectric elements in the ultrasound probe are directly above the backing 126, with respect to the propagation direction 101. The backing 126 may be configured to absorb ultrasonic waves directed from the piezoelectric element 102 in a direction opposite of the direction indicated by arrows 107 and attenuate stray ultrasonic waves deflected by the outer housing of the ultrasound probe. A bandwidth of the ultrasonic signal, as well as the axial resolution, may be increased by the backing 126.

A piezoelectric transducer (PZT) probe may provide high penetration into a target as well as high frequency and transient responses, enabling high resolution data to be obtained. However, a type of piezoelectric element included in the probe may operate within a frequency bandwidth that constrains use of the probe to a particular application. For example, probe with a low central frequency piezoelectric element may be used to produce ultrasound images of deep tissues or organs but may not provide sufficient flaw resolution or thickness measurement capabilities. Thus, use of piezoelectric transducer probes for a variety of applications may demand access to multiple probes with different piezoelectric elements.

In contrast, a capacitive micromachined ultrasonic transducer (CMUT) probe, when used in ultrasonic applications, may offer broader bandwidth as well as more efficient fabrication, due to construction of the CMUTs on silicon via micromachining techniques. The broader CMUT bandwidth enables the CMUT probes to achieve greater axial resolution than the PZT probe. However, a sensitivity and penetration of the CMUT probe may be less than the PZT probe. Furthermore, CMUTs may be more prone to acoustic crosstalk than PZTs.

In one example, high penetration and broad bandwidth may be provided in a PZT probe by adapting the PZT probe with transducers equipped with piezo-composite elements formed from more than one type of sub-element, each sub-element being a different type of piezoelectric material or a similar type of piezoelectric material but with different resonance frequencies. In some examples, the sub-element may include other materials used for acoustic signal generation and reception, such as single crystals. By combining piezoelectric sub-elements with different resonance frequencies into one transducer, an array of multi-frequency piezoelectric elements may be provided. Each of the multi-frequency elements may have a distinct frequency, depending on relative proportions of the sub-elements, allowing the multi-frequency elements to transmit and receive signals over a wider range of frequencies in comparison to a single element transducer probe.

Furthermore, the array may be configured such that a positioning and orientation of the sub-elements are not tied to a dicing pattern of the array to form individual piezo-composite elements. In other words, physical characteristics such as sub-element angle, kerf width, sub-element width, etc., may be selected independent of the dicing pattern. As a result, a range of frequency distribution is broadened, allowing a wafer formed of the piezo-composite elements to be used for different transducer probes.

For example, as shown in FIG. 2, an example of a first matrix 200 with multi-frequency elements may be a homogeneous bi-dimensional array, such as a butterfly-type matrix array, with homogeneous multi-frequency elements 202. The first matrix 200 may represent an arrangement of the elements 202 within a transducer of an acoustic stack, such as the acoustic stack 100 of FIG. 1. At least one of the acoustic stack may be incorporated within a PZT probe. The first matrix 200 is shown oriented along the elevation direction 105 and the azimuth direction 103.

Each of the elements 202 includes a first sub-element 204 and a second sub-element 206. As an example, the first sub-element 204 may be a higher frequency element and the second sub-element 206 may be a lower frequency element. The elements 202 may be spaced apart from one another and thereby electrically insulated from adjacent elements 202. Each of the elements 202 may be coupled to an electrical circuit 208 to enable application of a voltage to induce deformation of each of the elements 202. Furthermore, each of the elements 202 may transmit an individual signal, as induced by deformation. It will be noted that each of the elements 202 may couple to the electrical circuit 208 but only the bottom row of elements 202 are shown directly coupled to the electrical circuit 208 in FIG. 2 for brevity.

The first matrix 200 may be coupled to other layers of the acoustic stack, e.g., an acoustic lens, a backing, etc., as shown in FIG. 1. As a result of forming the elements from the first sub-element 204 and the second sub-element 206, the elements may transmit and/or receive across a wide range of frequencies. For example, the first sub-element 204 may have a central (e.g., resonance) frequency of 2.0 MHz and the second sub-element 206 may have a central frequency of 15 MHz. By combining the first sub-element 204 with the second sub-element 206 with equal relative proportions, the elements 202 may transmit and/or receive ultrasonic signals across a wider range of frequencies than either the first sub-element 204 or the second sub-element 206 alone.

With respect to fabrication, however, alignment of an array pattern, e.g., such as a grid for a 2D matrix, used to dice an acoustic stack to form individual elements of the transducer array may demand time-consuming alignment of the array pattern with the sub-elements. As described herein, a fabrication efficiency of a multi-frequency transducer array may be increased while enhancing a resolution of the multi-frequency transducer array by arranging the sub-elements at an angle relative to the azimuth direction in the acoustic stack. As a result, greater control over a performance of a resulting multi-frequency transducer is enabled in a low cost manner.

As an example, a piezo-composite 300 is depicted in FIGS. 3-5 overlaid with a first dicing pattern 302, a second dicing pattern 402, and a third dicing pattern 502, respectively, with the dicing patterns outlined with black lines. The same piezo-composite 300 is shown in FIGS. 3-5 as a representation of how one piezo-composite may be used in various types of transducer arrays to provide different effects on acoustic performance.

The first dicing pattern 302 of FIG. 3 may correspond to a 1D transducer array. The piezo-composite 300 includes a first piezoelectric material 304, or sub-element 304, which may be a low frequency piezoelectric material, and a second piezoelectric material 306, or sub-element 306, which may be a high frequency piezoelectric material (e.g., having a higher frequency than the first sub-element 304). In other examples, however, more than two sub-elements may be incorporated into the piezo-composite. The first and second sub-elements 304, 306 may be arranged in an alternating pattern, oriented parallel with one another and separated by kerfs 310. The kerfs 310 extend parallel with the sub-elements at a similar angle relative to the azimuth direction 103. In one example, the angle may be 30 degrees. In other examples, the angle may be anywhere between 0 and 90 degrees.

The first dicing pattern 302 divides the piezo-composite 300 into elements 308, each element 308 having a first portion formed of the first sub-element 304 and a second portion formed of the second sub-element 306. Relative proportions of the sub-elements may differ amongst the elements 308 due to non-alignment of the sub-elements with the first dicing pattern 302.

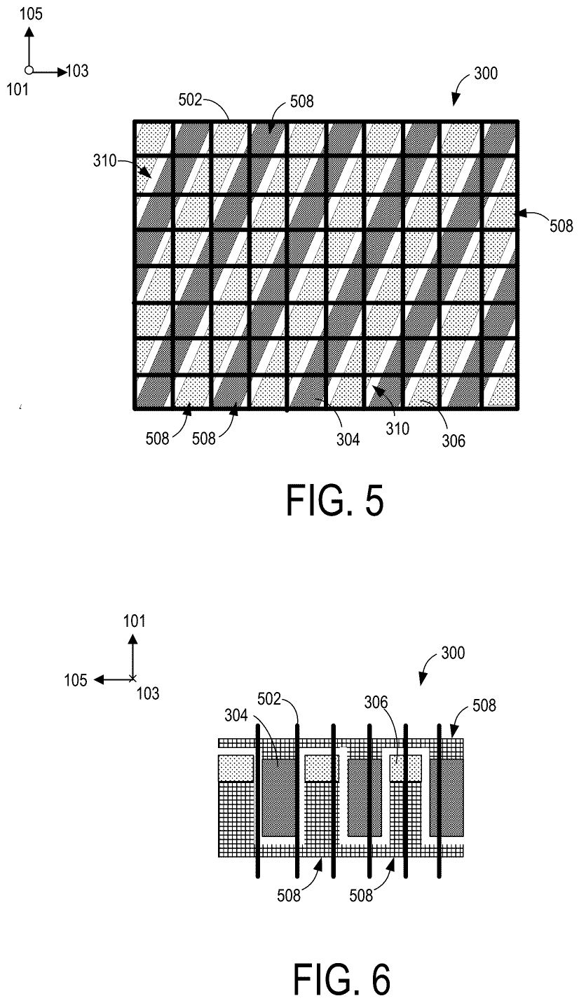

The second dicing pattern 402 of FIG. 4 may correspond to a 1.25 transducer array and the third dicing pattern 502 of FIG. 5 may correspond to a matrix 2D transducer array. Elements 408 defined by the second dicing pattern 402, as well as elements 508 defined by the third dicing pattern 502, may similarly include varying proportions of the first sub-element 304 and the second sub-element 306. As well, the second and third dicing patterns 402, 502 may not be aligned with orientations of the first and second sub-elements 304, 306. When the piezo-composite 300 is fabricated via a large-scale, wafer approach, a wafer including the first and second sub-elements 304, 306 may be singulated to form one or more of the transducer arrays of FIGS. 3-5. In other words, a common wafer may be utilized for different transducer probe types by applying different dicing patterns to acoustic stacks formed by singulating the wafer.

In some instances, increasing a difference in relative proportions of the sub-elements, e.g., an element composition, may be desirable to provide a transducer array with broadband capabilities that is able to transition smoothly between frequencies and provide high resolution. Decreasing a size of the element relative to dimensions of the sub-elements may increase a compositional variability between the elements. For example, the third dicing pattern 502 of FIG. 5 may have smaller element dimensions than the second dicing pattern 402 of FIG. 4, which, in turn, may have smaller element dimensions than the first dicing pattern 302 of FIG. 3.

The elements 508 of FIG. 5 may demonstrate greater variability in element composition than either of the elements 408 of FIG. 4 or the elements 308 of FIG. 3. Thus, a repetition frequency of element composition may be reduced as the element size is reduced with respect to a given piezo-composite. In some examples, a size of the element relative to dimensions of the sub-elements may be selected to achieve a target repetition frequency, as described further below.

Acoustic properties may also be controlled by selecting target thicknesses for each of the sub-elements, according to a desired effect. A ratio of a width to a thickness of a given element, the width defined along the azimuth direction 103 and the thickness defined along the propagation direction 101, may define a form factor of the element. For example, a variability in relative proportions of the first sub-element 304 and the second sub-element 306 resulting from the third dicing pattern 502 of FIG. 5 is illustrated in FIG. 6 in a side view of an acoustic stack having the third dicing pattern 502. A thickness of the first sub-element 304, the thickness defined along the propagation direction 101, may be greater than a thickness of the second sub-element 306. The elements 508 may therefore consistently have a greater proportion of the second sub-element 306 versus the first sub-element 304 which may allow the corresponding piezo-composite transducer probes to be more suitable for high resolution imaging applications while still having therapy capabilities. Conversely, by fabricating a piezo-composite with a low frequency sub-element having a greater thickness than a high frequency sub-element, the corresponding transducer probe may be more suitable for delivering therapy.

Acoustic properties of a multi-frequency transducer array may also be tuned by optimizing various physical characteristics of a piezo-composite. For example, a pitch of at least one of the sub-elements of the piezo-composite may be modified, as shown in FIG. 7. The piezo-composite 300 of FIG. 305 is depicted in FIG. 7, having the first and second sub-elements 304, 306 as described above. A pitch 702 of the sub-elements may be a distance, in microns, between a sub-element and a nearest sub-element of a same type. By adjusting the pitch relative to a given dicing pattern, such as any of the dicing patterns of FIGS. 3-5, a repetition frequency of the elements in the transducer array may be varied. For example, by decreasing the repetition frequency, e.g., by increasing differences between element composition such that fewer elements have the same composition, greater resolution for imaging and therapeutic delivery may be achieved.

As an example, for a given element width 704, the element width 704 defined along the azimuth direction 103, the pitch 702 may be adjusted relative to the element width 704. The elements of a diced array may become more homogeneous as the pitch 702 decreases. In addition, by maintaining the pitch 702 (and therefore sub-element width) small relative to the thickness of the respective sub-element, a form factor of the piezo-composite may be smaller than a form factor of the element, as described in Equation 1.

$${W}_{c o m p} / {T}_{p i e z o} << {W}_{e l e m e n t} / {T}_{p i e z o} \qquad ( 1 )$$

In Equation 1, W_{comp} is the width of the piezo-composite based on the pitch, Wₑₗₑₘₑₙₜ is the width of the element (as determined by a dicing pattern), and T_{piezo} is the overall thickness of the piezo-composite. By maintaining the form factor of the piezo-composite small relative to the form factor of the element, lateral spurious modes may be suppressed. As a result, a frequency response may be smoother which mitigates generation of a notch in a corresponding frequency response band, while reducing ringdown.

However, when the pitch 702 is equal to or larger than the element width 704, the corresponding elements may become more varied in composition relative to one another as the pitch 702 increases. While more homogeneous elements may suppress lateral spurious modes, elements with greater compositional variations within a given transducer array may provide an averaging effect over the different elements. As a result, a radiated beam profile of a transducer incorporating the piezo-composite may be improved, along with reduced undesirable radiating side lobes (grating lobes). The pitch may therefore be selected according to a desired application and effect on a performance of the transducer probe.

For example, if dimensions of the elements are relatively large, a relatively small pitch may be desirable to increase a homogeneity between the elements. Conversely, if the element dimensions are relatively small, decreasing the pitch to achieve a desired homogeneity may be impractical. As such element homogeneity may be selectively adjusted by varying a composition of the elements. Further, decreasing composite pitch may effectively improve form factor in instances where spurious lateral modes are present. However, when undesirable grating lobes are present, varying the element composition may be preferred over varying the pitch.

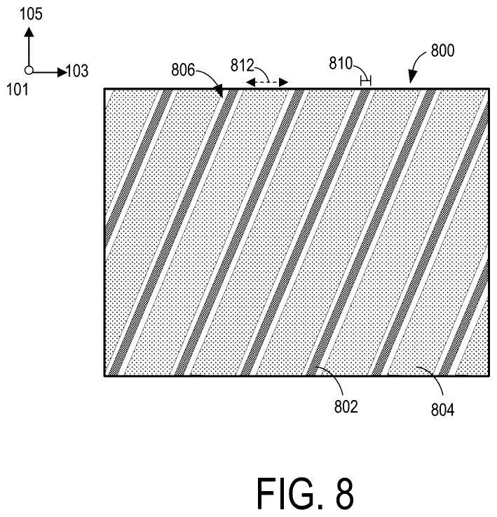

In another example, proportions of each sub-element may be varied relative to one another to achieve desired acoustic properties of the piezo-composite. For example, as shown in FIG. 8, a piezo-composite 800 with different proportions of a first sub-element 802 and a second sub-element 804 is shown. The first sub-element 802 and the second sub-element 804 may correspond to a low frequency and a high frequency material, respectively, and may be separated by kerfs 806. The sub-elements may be angled with respect to each of the azimuth direction 103 and the elevation direction 105 such that the sub-elements are not parallel with either direction but are parallel with one another.

A width 810 of the first sub-element 802 is narrower than a width 812 of the second sub-element 804. In one example, a fraction of the piezo-composite 800 formed by the first sub-element 802 may be 30% while a fraction of the piezo-composite 800 formed by the second sub-element 804 may be 70%. In contrast, the piezo-composite 300 of FIGS. 3-7 may be formed of 50% of the first sub-element 304 and 50% of the second sub-element 306. By varying the relative fractions of the sub-elements, a performance of a resulting transducer probe may be tuned for specific applications, in a similar manner as described above with respect to a form factor of the sub-elements and a form factor of the elements. For example, for applications relying more heavily on imaging, the fraction of the second sub-element 804 may be increased whereas for applications where delivery of treatment is prioritized, the fraction of the first sub-element 304 may be increased.

Furthermore, varying the fractions such that the sub-element proportions are unequal may result in greater inhomogeneity of composition amongst elements of the piezo-composite 800, as defined by dicing. Therefore, in examples where maintaining a pitch of the piezo-composite 800 small is desirable (e.g., such that a form factor of the piezo-composite 800 is smaller than a form factor of an element in a resulting transducer array), variations between element compositions may be increased by increasing a difference between the sub-element fractions.

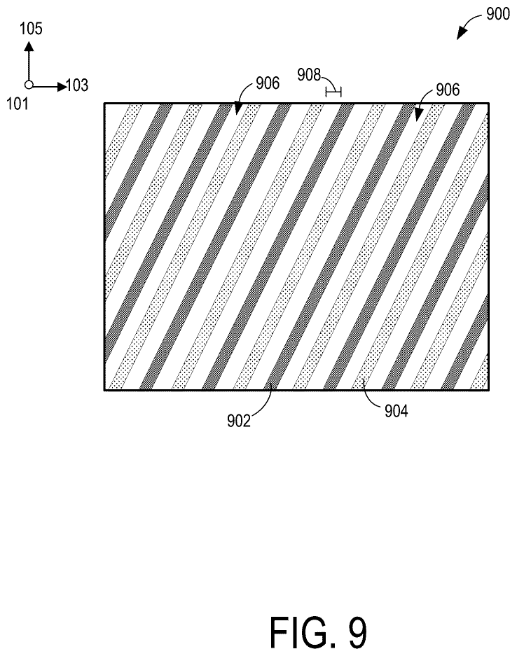

In instances where equal fractions of sub-elements of a piezo-composite is desirable, a repetition frequency of element composition may instead be decreased by adjusting a kerf width of the piezo-composite. For example, as shown in FIG. 9, a piezo-composite 900 may have a first sub-element 902 and a second sub-element 904, where both sub-elements are oriented at an angle to each of the azimuth direction 103 and the elevation direction 105 and parallel to one another. The sub-elements form equal fractions of the piezo-composite 900 of FIG. 9 but may have different relative proportions in other examples, when equal fractions of the sub-elements are not demanded.

The sub-elements may be separated by kerfs 906. A width 908 of the kerfs 906 are larger, for example, than a width of the kerfs 310 of the piezo-composite 300 of FIGS. 3-7. Assuming that a width of the first sub-element 902 is similar to a width of the first sub-element 304 of FIGS. 3-7 and a width of the second sub-element 904 is similar to a width of the second sub-element 306 of FIGS. 3-7, the wider kerfs 906 of the piezo-composite 900 of FIG. 9 may result in greater variation in element composition across a corresponding transducer array. For example, given a same element size for each of the piezo-composite 300 and the piezo-composite 900, the piezo-composite 900 of FIG. 9 may demonstrate decreased repetition frequency of element composition without demanding changes to sub-element proportion or width.

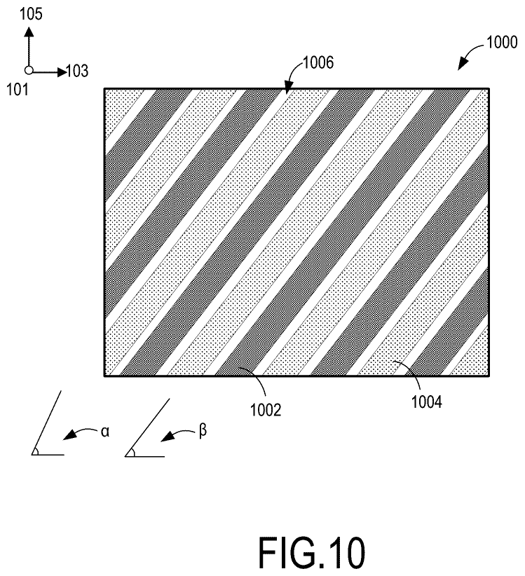

A repetition frequency of element composition may also be decreased by varying a sub-element angle of a piezo-composite. For example, as shown in FIG. 10, a piezo-composite 1000 may include a first sub-element 1002 and a second sub-element 1004 separated by kerfs 1006. The sub-elements, as shown in previous examples, are arranged in an alternating motif at an angle β relative to the azimuth direction 103. The angle β may be smaller than an angle α of the sub-elements of the piezo-composite 300 of FIGS. 3-7, which is indicated in FIG. 7 and reproduced in FIG. 10 for convenience. As an example, the angle α may be 80 degrees while the angle β may be 60 degrees.

When a common dicing pattern is applied to each of the piezo-composites 300 and 900, a greater difference in element composition between neighboring elements may be demonstrated by the piezo-composite 900. The repetition frequency of element composition may be decreased by decreasing the sub-element angle (with respect to the azimuth direction 103) while the repetition frequency of a transducer array may be increased by increasing the sub-element angle. However, when the sub-element angle reaches 0, the sub-elements may become more homogeneous. Decreasing the sub-element angle to a minimum angle that remains greater than 0 may provide maximum variations in element composition with respect to effects of sub-element angle. For example, repetition of a specific subset of the sub-elements may be enabled based on the sub-element angle. As an example, as the sub-element angle is increased, a periodicity of element composition may be increased. The sub-element angle may therefore be selected to prioritize either variation between consecutive elements or to introduce periodicity into multi-frequency patterns of the transducer array for specific groups of elements. An operating frequency range of the transducer may thereby be optimized according to end-use.

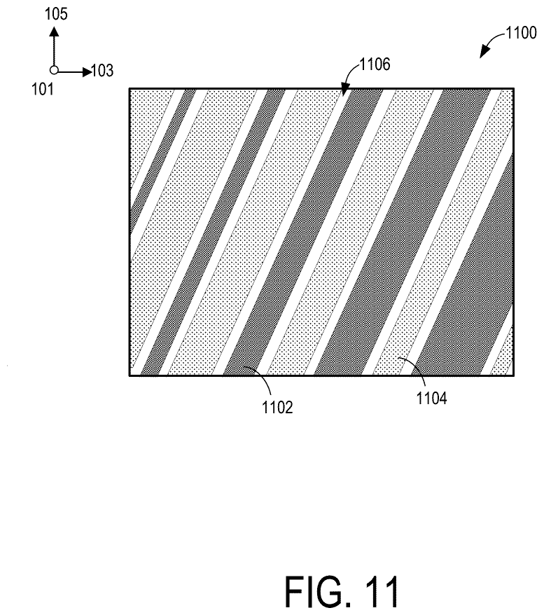

In yet another example of transducer probe optimization based on adjustment to piezo-composite attributes, greater inhomogeneity amongst elements of the transducer probe may also be enabled by varying a width of a sub-element within a piezo-composite. For example, as shown in FIG. 11, a piezo-composite 1100 includes a first sub-element 1102 and a second sub-element 1104, the sub-elements arranged in an alternating pattern and separated by kerfs 1106. Further, the sub-elements and the kerfs 1106 extend across the piezo-composite 1100 at an angle relative to the azimuth direction 103 and the elevation direction 105, which may be an angle between 0 and 90 degrees relative to either direction.

The first sub-element 1102 may increase in width sequentially, the width defined along the azimuth direction 103, to the right of FIG. 11 while the second sub-element 1104 may decrease sequentially in width to the right. As such, each of the first sub-element 1102 has a different width from one another while each of the second sub-element 1104 has a different width from one another within the piezo-composite 1100. The varying widths of each sub-element introduces greater inhomogeneity amongst elements of a corresponding transducer array (e.g., according to an applied dicing pattern). It will be appreciated that the sequential change in sub-element width depicted in FIG. 11 is a non-limiting example and, in other examples, the widths of a sub-element may vary instead in a non-sequential, non-linear manner. Further, in some examples, only a portion of the sub-elements included in a piezo-composite may vary in width. In other words, one or more of the sub-elements may have variable widths while one or more of the sub-elements may be uniform widths. In addition, variation in width of a given sub-element may occur in a random manner across the piezo-composite.

As described above, a piezo-composite, such as any of the piezo-composites of FIGS. 3-11, may be both formed and incorporated into a wafer-scale acoustic stack for a transducer probe via a wafer approach. The wafer approach enables large scale manufacturing of a piezo-composite acoustic stack which may be singulated into individual acoustic stacks for transducer probes. Examples of wafer-scale acoustic stacks are illustrated in FIGS. 12 and 14. Each of a first wafer-scale acoustic stack 1200 and a second wafer-scale acoustic stack 1400 of FIGS. 12 and 14, respectively, may include two sub-elements arranged in an alternating pattern, e.g., as shown in FIGS. 3-11, with the sub-elements separated by kerfs and arranged at an angle relative to the azimuth direction 103 and the elevation direction 105. The sub-elements may be piezoelectric transducer materials, single crystals, or other materials capable of generating and receiving acoustic signals. Further, the sub-elements may or may not be of a same type of sub-element, and in other examples, may incorporate more than two sub-elements. In addition, the wafer-scale acoustic stacks may include additional layers and components not described herein for brevity.

Turning first to FIG. 12, the first wafer-scale acoustic stack 1200 may be an interdigitated structure including a piezo-composite 1201 formed of a first sub-element 1202 and a second sub-element 1204 which are separated by kerfs 1206. The first sub-element 1202 may be incorporated in a first comb structure 1208 which may be an acoustic stack formed of the first sub-element 1202, a matching layer 1210, and a backing layer 1212, which may be stacked as shown in FIG. 12 and coupled to a first substrate 1214. The first substrate 1214 may be formed of a conductive material such as graphite, aluminum, steel, etc. The matching layers 1210 may be formed of one or more conductive materials such as a gold-coated substrate, flex conductive materials, etc., and may be the same material or different materials. The backing layer 1212 may be an optional layer (or layers when formed of more than one layer) that is formed of one or more of an application specific integrated circuit board (ASIC), a flex conductive material, a printed circuit board (PCB), a metal, etc. Initially, the layers of the first comb structure 1208 may be laminated to one another using a conductive adhesive, as one example, to form a first acoustic block.

The first acoustic block may be diced to form the first comb structure 1208, where the first acoustic block may be diced according to match dimensions of a second comb structure 1216 of the first wafer-scale acoustic stack 1200. For example, as shown in FIG. 12, the first comb structure 1208 and the second comb structure 1216 may be diced to have complementary geometries such that teeth of the first comb structure 1208 fit into recesses in the second comb structure 1216 and teeth of the second comb structure 1216 fit into recesses of the first comb structure 1208. As such, the comb structures, when combined, form an overall interdigitated construction of the first wafer-scale acoustic stack 1200. The first and second comb structures 1208, 1216 may thereby be meshed together to form a layered block structure of the first wafer-scale acoustic stack 1200.

The second comb structure 1216 includes the second sub-element 1204, a matching layer 1218, an optional backing layer 1220, and a second substrate 1222 and may be initially formed as a second acoustic block that is diced as described above. The second substrate 1222 may be formed of one of the materials listed above for the first substrate 1214, the matching layer 1218 may be formed of any of the materials described above for the matching layers 1210 of the first comb structure 1208, and the backing layer 1220 may be formed of any of the materials described above for the backing layer 1212 of the first comb structure 1208.

When the first comb structure 1208 and the second comb structure 1216 are combined to form the first wafer-scale acoustic stack 1200, the kerfs 1206 may be gaps between the comb structures, e.g., between sub-elements of the piezo-composite 1201, and may be filled with a nonconductive and electrically insulating filler, such as silicone, resin, or epoxy. The first wafer-scale acoustic stack 1200 may be ground at each of a first face 1224 and a second face 1226 to remove a first portion of a thickness of the first wafer-scale acoustic stack 1200, as indicated by dashed rectangle 1228, and a second portion of the thickness of the first wafer-scale acoustic stack 1200, as indicated by dashed rectangle 1228, respectively. By removing the first portion of the thickness of the first wafer-scale acoustic stack 1200, the matching layers 1210 and 1218 may be exposed (e.g., to air or surrounding atmosphere) and by removing the second portion of the thickness of the first wafer-scale acoustic stack 1200, at least the backing layer 1212 of the first comb structure 1208 may be exposed. Ground recovery may be enabled by removing the first and second portions of the first and second substrates 1214, 1222 that cover the respective matching and backing layers.

In the example of FIG. 12, the piezo-composite may be formed from sub-elements with already associated matching and backing layers, thereby precluding subsequent lamination of the matching and backing layers to the piezo-composite. In other words, each sub-element has pre-existing matching and backing layers, which are incorporated into the wafer-scale acoustic stack upon combining of the sub-elements into the piezo-composite. In some instances, it may be desirable to fabricate a piezo-composite without matching and backing layers already laminated to structures that are diced and combined to form the piezo-composite. For example, as shown in FIGS. 13-14, a second wafer-scale acoustic stack 1400 (as shown in FIG. 14) may be formed from a piezo-composite 1300 including a first sub-element 1302 and a second sub-element 1304. The second wafer-scale acoustic stack 1400 is also an interdigitated structure.

The first sub-element 1302 may be incorporated in a first substrate 1306, which may be formed of a conductive material as described above, and the first substrate 1306 may be diced into a first comb structure 1308. The second sub-element 1304 may be incorporated in a second substrate 1310, similarly formed of a conductive material as described above, and the second substrate 1310 may be diced into a second comb structure 1312. The first and second comb structures 1308, 1312 may have complementary geometries that allow the structures to be combined as shown in FIG. 13 and described above with respect to the first and second comb structures 1208, 1216 of FIG. 12. When the comb structures are combined to form the piezo-composite 1300 of FIGS. 13-14, the first and second sub-elements 1302, 1304 may be separated by kerfs 1314 which may be filled with a non-conductive, electrically insulating filler.

A first portion of a thickness of the piezo-composite 300, as indicated by dashed rectangle 1316, and a second portion of the thickness of the piezo-composite 300, as indicated by dashed rectangle 1318 may be removed by grinding. The first and second portions may be located at opposite faces of the piezo-composite 300 and may remove layers of the first substrate 1306 and the second substrate 1310 covering the first and second sub-elements 1302, 1304, respectively. For example, removing the first portion may expose surfaces of the first sub-element 1302 at a first face 1320 of the piezo-composite 300 to air or surrounding atmosphere and removing the second portion may expose surfaces of the second sub-element 1304 at a second face 1322 of the piezo-composite 300 to air or surrounding atmosphere.

The second wafer-scale acoustic stack 1400 may be formed after grinding of the piezo-composite 300 by coupling matching layers 1402 to the first face 1320 of the piezo-composite 1300 and coupling a backing layer 1404 to the second face 1322 of the piezo-composite 1300. It will be noted that the piezo-composite 1300 is oriented upside-down in FIG. 14 relative to FIG. 13. The matching layers 1402 may be formed of a same or different material, which may include one or more conductive materials such as a gold-coated substrate, flex conductive materials, etc. The backing layer 1404, which may be an optional layer, may be formed of one or more layers including an ASIC, a flex conductive material, a printed circuit board (PCB), a metal, etc.

The second wafer-scale acoustic stack 1400 may allow matching and backing layers to be common amongst all elements of the second wafer-scale acoustic stack 1400. In contrast, the first wafer-scale acoustic stack 1200 of FIG. 12 may enable matching and backing layers specific to each sub-element type to be incorporated therein. The first wafer-scale acoustic stack 1200 may be preferred when the sub-element frequencies are far apart, such as when a frequency of one sub-elements is below 1 MHz for providing therapy and another of the sub-elements is in a MHz range for image acquisition. As well, the first wafer-scale acoustic stack 1200 may be preferred for applications where coupling of the sub-elements (e.g., coupling through a matching layer) is not desired. In contrast, the second wafer-scale acoustic stack 1400 may be preferred when it is desirable to increase the coupling between the sub-elements and the frequencies of the sub-elements are relatively close in range. For example, the second wafer-scale acoustic stack 1400 may be preferred when one sub-element transmits signals at a first frequency, f₀ and another of the sub-elements transmits signals at a second frequency equal to 2f₀, such as for harmonic imaging.

In another example, grinding of a piezo-composite to form an acoustic may be precluded by dicing sub-wafers of each sub-element in a side-ways manner, as shown in FIG. 17, rather than along the propagation direction 101, as shown in FIGS. 12-14. For example, as depicted in FIG. 17, a third wafer-scale acoustic stack 1700 may include a first comb structure 1702 and a second comb structure 1704. The first comb structure 1702 may include a first sub-element supported by a first substrate and the second comb structure 1704 may similarly include a second sub-element supported by a second substrate.

In some examples, matching and/or backing layers may be coupled to each of the first comb structure 1702 and the second comb structure 1704 prior to dicing. In other examples, the matching and/or backing layers may be coupled to the wafer-scale acoustic stack 1700 after dicing and combining the first and second comb structures 1702, 1704. For example, the first comb structure 1702 may be diced along a plane formed by the azimuth direction 103 and the elevation direction 105 (e.g., along the x-y plane), rather than along the propagation direction as shown in FIGS. 12-14. As such, dicing along the x-y plane is referred to as side-ways dicing whereas dicing along the z-axis 101 (e.g., the propagation direction 101) is referred to as top-down dicing.

The dicing of the first comb structure 1702 may be angled to form first teeth 1706 that are angled with respect to each of the azimuth direction 103 and the elevation direction 105. The angling of the first teeth 1706 shown in FIG. 17 is non-limiting, however, and each of the comb structures may be diced at any desired angle along the x-y plane, in a complementary manner to one another. For example, the comb structures may be diced parallel to the elevation direction 105, parallel to the azimuth direction 103, or along any angle in between.

The second comb structure 1704 may be diced along the x-y plane and along an angle in common with the first comb structure 1702. For example, the second comb structure 1704 may be diced to have second teeth 1708 that extend parallel with the first teeth 1706 of the first comb structure 1702. The second comb structure 1704 may be diced from an opposite direction, however, to allow the second comb structure 1704 to have a shape that is complementary to the first comb structure 1702. In other words, the first teeth 1706 may fit in spaces between the second teeth 1708, and the second teeth 1708 may fit in spaces between the first teeth 1706, when the first and second comb structures 1702, 1704 are meshed together, as indicated by arrows 1710. Widths of each of the first and second teeth may be selected according to a desired pitch of the sub-elements, as well as to a target element composition (e.g., relative fractions of the sub-elements).

By dicing the comb structures side-ways instead of top-down, grinding of upper and lower portions, such as the portions indicated by the dashed rectangles 1228 and 1230 in FIG. 12 and the dashed rectangles 1316 and 1318 of FIG. 13, of the third wafer-scale acoustic stack 1700 is therefore not demanded. As a result, a fabrication process for the third wafer-scale acoustic stack 1700 may be simplified relative to fabrication of wafer-scale acoustic stacks relying on top-down dicing.

A wafer-scale acoustic stack may be of a sufficient size to be divided into multiple transducer arrays, where each of the transducer arrays is a portion of the wafer scale acoustic stack. For example, as illustrated in FIG. 15 in a representative diagram, a wafer-scale acoustic stack 1500 (hereafter, wafer 1500), as indicated by a dashed rectangle, may be diced according to a grid pattern to form elements 1502 with a square geometry as shown from a top view 1501 of the wafer 1500. The wafer 1500 may be, for example, configured similarly to the first, second, or third wafer-scale acoustic stacks 1200, 1400, or 1700 of FIG. 12, 14, or 17 and may incorporate a piezo-composite 1505. As shown in a side view 1503 of transducer arrays 1504 obtained from the wafer 1500, the wafer 1500 may be diced from an upper face 1506 downwards, relative to the propagation direction 101, into a portion of a thickness of a backing layer 1508 of the wafer 1500.

The wafer 1500 may undergo singulation to divide the wafer 1500 into the individual transducer arrays 1504. The individual transducer arrays 1504 may be similarly sized, as shown in FIG. 15, or may be differently sized, according to desired array dimensions, or to obtain transducer arrays having a target quantity of the elements 1502. In addition, the transducer arrays 1504 may be diced to form a same type of array (as shown in FIG. 15) or diced to form different types of arrays. In some examples, the transducer arrays 1504 may be diced according to target dicing patterns after singulation, rather than before. The transducer arrays 1504 may be incorporated into transducer probes and used for generating and receiving acoustic signals.

A method 1600 for fabricating a transducer array having a piezo-composite, such as the piezo-composites shown in FIGS. 3-15, and 17, via a wafer approach is depicted in FIG. 16. The piezo-composite may include various types of sub-element materials able to both generate and receive acoustic signals. At 1602, the method includes forming acoustic blocks for each sub-element to be incorporated into the piezo-composite, which may include two or more sub-elements. For example, an acoustic block may be constructed from a conductive substrate to which a sub-element may be incorporated by, for example, lamination. In some examples, the acoustic blocks may include matching and/or backing layers. The acoustic blocks may be diced at 1604 into comb structures with complementary geometries, such as the comb structures shown in FIGS. 12-14.

At 1606, the method includes combining the comb structures formed from the diced acoustic stacks to produce an interdigitated structure. The interdigitated structure may be similar in configuration to, for example, the wafer-scale acoustic stacks 1200, 1400, and 1700 of FIGS. 12, 14, and 17, respectively. A piezo-composite wafer, with or without matching and backing layers, may be formed by combining, e.g., meshing, the comb structures. When the comb structures include matching and/or backing layers, an acoustic stack is formed.

In instances where the piezo-composite wafer is assembled from comb structures that are diced top-down (e.g., as shown in FIGS. 12-14), the piezo-composite wafer may be ground at 1608 of the method. For example, a first portion of a thickness of the piezo-composite wafer may be removed at a first face of the wafer and a second portion of the thickness may be removed at a second, opposite face of the wafer. By removing the portions of the piezo-composite wafer at the first and second faces, either matching and backing layers of the piezo-composite may be exposed, when the comb structures of the piezo-composite wafer include the matching and backing layers, or surfaces of the sub-elements may be exposed, when the comb structures do not include the matching and backing layers. In examples, however, where the comb structures are diced side-ways, as shown in FIG. 17, grinding at 1608 is omitted.

The method may optionally include coupling the matching and/or backing layers at 1610. For example, if one or more of the matching and backing layers are not included in the comb structures for each sub-element, the matching and backing layers may be attached to opposite faces, e.g., the first and second faces, respectively, of the piezo-composite wafer. As an example, the matching and backing layers may be coupled via lamination. Upon adding the matching and backing layers, an acoustic stack is formed.

At 1612, the method includes dicing and singulating the piezo-composite wafer. For example, a dicing pattern may be used to dice the wafer according to target dimensions and geometry of elements. By dicing the wafer, the elements are separated and electrically insulated from one another, where each element may be part of an individual electrical circuit. Upon singulation, the piezo-composite wafer may be cut into individual acoustic stacks, each acoustic stack forming a transducer array that may be incorporated into a transducer probe. The wafer may be diced before singulation or after singulation.

In this way, a multi-frequency transducer array may be provided with high resolution, extended usable bandwidth, and smooth frequency response. A manufacturing of the multi-frequency transducer array may be achieved at low cost and with increased efficiency by precluding alignment of sub-elements of the array with a target dicing pattern. By removing a dependency of sub-element orientation on dicing pattern, attributes of the sub-elements may be adjusted to obtain desired performance characteristics of the multi-frequency transducer array. More specifically, by achieving greater inhomogeneity in element composition across the array, spurious and lateral modes may be suppressed during operation and ringdown of the transducer array may be reduced. As a result, fewer undesirable imaging artifacts may be observed.

The disclosure also provides support for a transducer array, comprising: a plurality of elements formed of two or more piezoelectric materials, the two or more piezoelectric materials having different resonance frequencies, wherein the two or more piezoelectric material types are oriented independent of a dicing pattern of the transducer array, the dicing pattern defining the plurality of elements. In a first example of the system, the plurality of elements have different compositions, and wherein the compositions comprise fractions of the two or more piezoelectric materials. In a second example of the system, optionally including the first example, the two or more piezoelectric materials are not aligned with the dicing pattern of the transducer array, and wherein the two or more piezoelectric materials are arranged at an angle relative to an azimuth direction of the transducer array. In a third example of the system, optionally including one or both of the first and second examples, the angle is 30 degrees. In a fourth example of the system, optionally including one or more or each of the first through third examples, the angle is between 0 and 90 degrees. In a fifth example of the system, optionally including one or more or each of the first through fourth examples, inhomogeneity of a composition of the plurality of elements increases when the angle is decreased to a minimum angle that is greater than 0 degrees. In a sixth example of the system, optionally including one or more or each of the first through fifth examples, a periodicity of composition amongst the plurality of elements is increased when the angle is increased. In a seventh example of the system, optionally including one or more or each of the first through sixth examples, an inhomogeneity of composition amongst the plurality of elements is adjusted by varying one or more of a pitch of the two or more piezoelectric materials, fractions of the two or more piezoelectric materials, a kerf width, an angle of the two or more piezoelectric materials, and widths of the two or more piezoelectric materials.

The disclosure also provides support for a piezo-composite acoustic stack, comprising: elements defined by a dicing pattern, the elements comprising a piezo-composite including a first piezoelectric material and a second piezoelectric material arranged in an alternating pattern and not aligned with the dicing pattern, wherein the piezo-composite acoustic stack is formed of a first comb structure having the first piezoelectric material and a second comb structure having the second piezoelectric material. In a first example of the system, the dicing pattern is configured to define the piezo-composite acoustic stack as one of a 1D, 1.25D, 1.5D, 1.75D or a matrix array. In a second example of the system, optionally including the first example, the first piezoelectric material and the second piezoelectric material have different resonance frequencies, and each of the elements has fractions of the first piezoelectric material and the second piezoelectric material determined based on physical characteristics of the piezo-composite. In a third example of the system, optionally including one or both of the first and second examples, the physical characteristics includes a pitch of the piezo-composite, and wherein when the pitch is equal to or greater than a width of the elements, inhomogeneity of element composition is increased. In a fourth example of the system, optionally including one or more or each of the first through third examples, when the fractions of the first and the second piezoelectric materials are varied, an optimization of the piezo-composite acoustic stacks for imaging versus therapy delivery is adjusted. In a fifth example of the system, optionally including one or more or each of the first through fourth examples, the physical characteristics includes a kerf width, and wherein when the kerf width is increased, an inhomogeneity of element composition is increased. In a sixth example of the system, optionally including one or more or each of the first through fifth examples, the physical characteristics includes an angle of the first and the second piezoelectric materials relative to an azimuth direction, and wherein the angle is varied, a repetition frequency of element composition is varied. In a seventh example of the system, optionally including one or more or each of the first through sixth examples, the physical characteristics includes a width of each of the first and the second piezoelectric materials across the piezocomposite, and wherein when the width of at least one of the first and the second piezoelectric materials is varied, an inhomogeneity of element composition is increased.

The disclosure also provides support for a method for fabricating a piezo-composite transducer array, comprising: dicing a first acoustic stack with a first piezoelectric material and a second acoustic stack with a second piezoelectric material to have complementary geometries, combining the first acoustic stack and the second acoustic stack to form an interdigitated structure, and defining a plurality of elements in the interdigitated structure by a dicing pattern, each of the plurality of elements having a composition comprising fractions of the first and the second piezoelectric materials, wherein the first piezoelectric material and the second piezoelectric material are arranged in the interdigitated structure at an angle relative to an azimuth direction, independent of the dicing pattern. In a first example of the method, a frequency bandwidth and resolution of the piezo-composite transducer array is tuned by adjusting one or more of a pitch of the first and the second piezoelectric materials, a relative fraction of the first and the second piezoelectric materials, a kerf width, the angle of the first and the second piezoelectric materials, and widths of the first and the second piezoelectric materials. In a second example of the method, optionally including the first example, the method further comprises: grinding the interdigitated structure to expose matching and backing layers of each of the first and the second acoustic stacks prior to defining the plurality of elements. In a third example of the method, optionally including one or both of the first and second examples, the method further comprises: prior to defining the plurality of elements, grinding the interdigitated structure to expose surfaces of the first piezoelectric material and surfaces of the second piezoelectric material and coupling at least one of a matching layer and a backing layer to the interdigitated structure when the interdigitated structure is formed of comb structures with teeth extending parallel with a propagation direction of the piezo-composite transducer array, and wherein at least one of the matching layer and the backing layer is coupled to the interdigitated structure without grinding the interdigitated structure when the interdigitated structure is formed of comb structures with teeth extending perpendicular to the propagation direction.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A transducer array, comprising:
a plurality of elements formed of two or more piezoelectric materials, the two or more piezoelectric materials having different resonance frequencies, wherein the two or more piezoelectric materials are oriented independent of a dicing pattern of the transducer array, the dicing pattern defining the plurality of elements.

2. The transducer array of claim 1, wherein the plurality of elements have different compositions, and wherein the compositions comprise fractions of the two or more piezoelectric materials.

3. The transducer array of claim 1, wherein the two or more piezoelectric materials are not aligned with the dicing pattern of the transducer array, and wherein the two or more piezoelectric materials are arranged at an angle relative to an azimuth direction of the transducer array.

4. The transducer array of claim 3, wherein the angle is 30 degrees.

5. The transducer array of claim 3, wherein the angle is between 0 and 90 degrees.

6. The transducer array of claim 3, wherein inhomogeneity of a composition of the plurality of elements increases when the angle is decreased to a minimum angle that is greater than 0 degrees.

7. The transducer array of claim 3, wherein a periodicity of composition amongst the plurality of elements is increased when the angle is increased.

8. The transducer array of claim 1, wherein an inhomogeneity of composition amongst the plurality of elements is adjusted by varying one or more of a pitch of the two or more piezoelectric materials, fractions of the two or more piezoelectric materials, a kerf width, an angle of the two or more piezoelectric materials, and widths of the two or more piezoelectric materials.

9. A piezo-composite acoustic stack, comprising:
elements defined by a dicing pattern, the elements comprising a piezo-composite including a first piezoelectric material and a second piezoelectric material arranged in an alternating pattern and not aligned with the dicing pattern, wherein the piezo-composite acoustic stack is formed of a first comb structure having the first piezoelectric material and a second comb structure having the second piezoelectric material.

10. The piezo-composite acoustic stack of claim 9, wherein the dicing pattern is configured to define the piezo-composite acoustic stack as one of a 1D, 1.25D, 1.5D, 1.75D or a matrix array.

11. The piezo-composite acoustic stack of claim 9, wherein the first piezoelectric material and the second piezoelectric material have different resonance frequencies, and each of the elements has fractions of the first piezoelectric material and the second piezoelectric material determined based on physical characteristics of the piezo-composite.

12. The piezo-composite acoustic stack of claim 11, wherein the physical characteristics include a pitch of the piezo-composite, and wherein when the pitch is equal to or greater than a width of the elements, inhomogeneity of element composition is increased.

13. The piezo-composite acoustic stack of claim 11, wherein when the fractions of the first and the second piezoelectric materials are varied, an optimization of the piezo-composite acoustic stack for imaging versus therapy delivery is adjusted.

14. The piezo-composite acoustic stack of claim 11, wherein the physical characteristics include a kerf width, and wherein when the kerf width is increased, an inhomogeneity of element composition is increased.

15. The piezo-composite acoustic stack of claim 11, wherein the physical characteristics include an angle of the first and the second piezoelectric materials relative to an azimuth direction, and wherein the angle is varied, a repetition frequency of element composition is varied.

16. The piezo-composite acoustic stack of claim 11, wherein the physical characteristics include a width of each of the first and the second piezoelectric materials across the piezo-composite, and wherein when the width of at least one of the first and the second piezoelectric materials is varied, an inhomogeneity of element composition is increased.

17. A method for fabricating a piezo-composite transducer array, comprising:
dicing a first acoustic stack with a first piezoelectric material and a second acoustic stack with a second piezoelectric material to have complementary geometries;
combining the first acoustic stack and the second acoustic stack to form an interdigitated structure; and
defining a plurality of elements in the interdigitated structure by a dicing pattern, each of the plurality of elements having a composition comprising fractions of the first and the second piezoelectric materials, wherein the first piezoelectric material and the second piezoelectric material are arranged in the interdigitated structure at an angle relative to an azimuth direction, independent of the dicing pattern.

18. The method of claim 17, wherein a frequency bandwidth and resolution of the piezo-composite transducer array is tuned by adjusting one or more of a pitch of the first and the second piezoelectric materials, a relative fraction of the first and the second piezoelectric materials, a kerf width, the angle of the first and the second piezoelectric materials, and widths of the first and the second piezoelectric materials.

19. The method of claim 17, further comprising grinding the interdigitated structure to expose matching and backing layers of each of the first and the second acoustic stacks prior to defining the plurality of elements.

20. The method of claim 17, further comprising, prior to defining the plurality of elements, grinding the interdigitated structure to expose surfaces of the first piezoelectric material and surfaces of the second piezoelectric material and coupling at least one of a matching layer and a backing layer to the interdigitated structure when the interdigitated structure is formed of comb structures with teeth extending parallel with a propagation direction of the piezo-composite transducer array, and wherein at least one of the matching layer and the backing layer is coupled to the interdigitated structure without grinding the interdigitated structure when the interdigitated structure is formed of comb structures with teeth extending perpendicular to the propagation direction.
